(19) United States
(12) Patent Application Publication
(10) Pub. No.: US 2007/0190191 A1
(43) Pub. Date: Aug. 16, 2007

(54) Use of geraniol in antitumoral therapy

(76) Inventors: Francis Raul, Schiltigheim (FR); Stephanie Carnesecchi, Strasbourg (FR); Francine Gosse, Bischheim (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/552,965

(22) PCT Filed: Apr. 15, 2003
(86) PCT No.: PCT/EP03/08208

§ 371 Date: Jul. 21, 2006

(51) Int. Cl. A61K 36/185; A61K 36/13

(52) U.S. Cl. 424/770; 424/725

(57) ABSTRACT

The present invention is relative to the use of geraniol for blocking the development and differentiation of tumor cells, and potentiating the cytotoxic effect of antitumoral drugs.

The present invention relates to the field of anti-tumoral therapy and more specifically to the sensitization to antitumoral drugs of tumoral cells resistant to chemotherapy.

A conventional approach in the field of cancer therapy is the “differentiation” therapeutic approach (Lotan, 1990), which is based on “the demonstration that cancer is reversible and that the transformed phenotype can be suppressed by cytostatic agents and by differentiation-inducing physiological and pharmacological agents” (Lotan, 1990, p. 3460). According to this approach, inducing the differentiation of cancer cells would result in a less malignant phenotype.

However, it has been reported that some differentiated cancer cells keep all their malignant potentiality. Among these, the best known is the human colon cancer cell line Caco-2 which spontaneously undergoes structural and functional enterocytic differentiation in culture at late confluency (Pinto et al., 1983). Phenotypic changes that occur after confluency include the formation of brush-border membranes and expression of intestinal hydrolases, which are markers of functional differentiation also found in enterocytes and human fetal colonocytes (Rousset, 1986). It has been shown that late postconfluent differentiated Caco-2 cells remain tumorigenic in nude mice, and that differentiated cells are able to dedifferentiate in vitro (Pandrea et al., 2000).

Similarly, recent results obtained with a human hepatoma cell line have shown that, as for Caco-2 cells, the differentiation process was reversible and did not prevent the cells from reentering the cell cycle (Glaise et al., 1998).

Furthermore, it has been observed that clusters of cells which express differentiation characteristics of enterocytes are present to variable extents in all colonic cancers “in situ” and that resistance to high concentrations of chemotherapeutic agents seems to be restricted to cells with this enterocytic phenotype (Lesuffleur et al., 1998). Previous studies have shown that exposure of HT29 cells to increasing concentrations of methotrexate or 5-fluorouracil completely eliminates undifferentiated cell types, preserving a population of differentiated cells with enterocytic phenotype (Lesuffleur et al., 1991). It has also been reported that Caco-2 differentiated cells are essentially resistant to butyrate treatment (Ho et al., 1994) and that the response of Caco-2 cells to butyrate depends on their phenotype (Mariadason et al., 2001).

Thus it appears that prevention of cell differentiation might be an important factor in the treatment of cancer, in particular in the case of tumors resistant to chemotherapy, such as those found frequently for instance in colon cancer.

The inventors have now found that geraniol, an acyclic monoterpene alcohol found in particular in lemongrass and aromatic herb oils, is a potent inhibitor of tumor cells differentiation, and increases the cytotoxicity of antitumoral drugs. In particular, they have found that combinations of geraniol with a cytotoxic antitumoral agent have an increased anti-proliferative activity and are more active than compositions containing only the individual components. In particular, the inventors have found significant anti-proliferative activity for concentrations of cytotoxic antitumoral agent which alone had no significant effect on the growth of the cells.

Geraniol was already known for its in vitro and in vivo antitumor activity against murine leukemia, hepatoma, and melanoma cells (Shoff et al., 1991; Yu et al., 1995; Burke et al., 1997). More recently, the team of the inventors has reported an antiproliferative effect of geraniol on human colon cancer cells (Carnesecchi et al., 2001).

However, the effects of geraniol on cell differentiation has never been studied. Actually, the blocking effect found by the inventors is quite unexpected, in view of the known properties of other antitumoral monoterpenes, such as limonene and perillyl alcohol, which have been reported as differentiation inducers (Shi and Gould, Cancer Lett, 95, 1-6 1995).

Thus, the present invention relates to the use of geraniol for blocking differentiation of tumor cells. In particular, the invention provides a method for blocking differentiation of tumor cells wherein said method comprises contacting said cells with geraniol.

According to a preferred aspect, the present invention relates to the use of geraniol for potentiating the cytotoxic effect of an antitumoral agent.

In particular, the invention provides a method for potentiating the cytotoxic effect of an antitumoral agent on tumoral cells, wherein said method comprises treating said cells with said antitumoral agent in combination with geraniol.

According to a preferred embodiment the invention, said method is used for treating a tumor in a mammal, preferably a human, and comprises administering to said mammal said antitumoral agent in combination with geraniol.

According to another preferred embodiment of the invention, at least a part of said tumoral cells are differentiated cells, resistant to chemotherapy. Examples of tumors containing such cells include, in a non-limitative way, colorectal cancers, hepatoma, digestive and aero-digestive cancers, prostate cancers. According to still another preferred embodiment of the invention, said antitumoral agent is selected from nucleoside analogs, such as 5-fluorouracil (5-FU), oral derivatives of fluorouracil (UFT, Capecitabine), camptothecin analogs such as irinotecan (CPT-11), platinium diammonocyclohexan (oxaliplatinium), taxol.

The invention also provides a therapeutic combination comprising a cytotoxic antitumoral agent and geraniol. Both components can be mixed together, in a same container, or alternatively, provided in separate containers. Generally, they will be associated with pharmaceutically acceptable carriers, solvents, or diluents.

Amounts of geraniol effective in vitro for blocking differentiation of tumor cells and potentiating the cytotoxic effect of an antitumoral agent preferably range from about 100 to about 500 μM.

For in vivo administration, effective amounts of geraniol will preferably range from about 50 to about 300 mg/kg body weight, more preferably from about 100 to about 200 mg/kg.

In view of the potentiating effect of geraniol disclosed by the present invention, amounts of the cytotoxic antitumoral agent may be reduced when compared to those employed when said cytotoxic agent is used alone. Generally, they will preferably range from about ½ to about 1/25 of the amounts usually used for the cytotoxic agent alone. For instance, in the case of 5-FU, the amounts administered in the context of a combination of the invention will preferably range from about 5 to about 50 mg/kg body weight, more preferably from about 10 to about 40 mg/kg body weight.

For the practice of the invention, the cytotoxic antitumoral agent and the geraniol may be administered together or separately. If they are administered separately, the administration may be simultaneous or alternate.

Administration may be oral, topical, or parenteral. Generally, parenteral administration will be preferred. This includes subcutaneous, intravenous, intramuscular, intraperitoneal or intratumoral administration, as well as infusion into a tumor-adjacent artery.

Of course, the dosages and administration methods indicated above are only given by way of example. As would be understood by those skilled in the art, the more appropriate dosages and modes of administration may vary according to the age and condition of the patient, the stage of the carcinoma, and other such factors, and will readily be determined by a skilled clinician.

Since the combinations of the present invention are efficient at far lower doses of cytotoxic antitumoral agent than those that would be used with the cytotoxic antitumoral agent alone, and since geraniol is essentially devoid of toxic side effects, the present invention allows to obtain anti-proliferative effects that would otherwise be achievable only by the use of doses of cytotoxic antitumoral agent which would be intolerable due to adverse side effects.

The present invention will be further illustrated by the examples which follows and the accompanying drawings, which illustrate the potentiating effects of geraniol on anti-proliferative activity of a cytotoxic antitumoral agent. It should be understood however that these examples are given only by way of illustration of the invention and do not constitute in any way a limitation thereof.

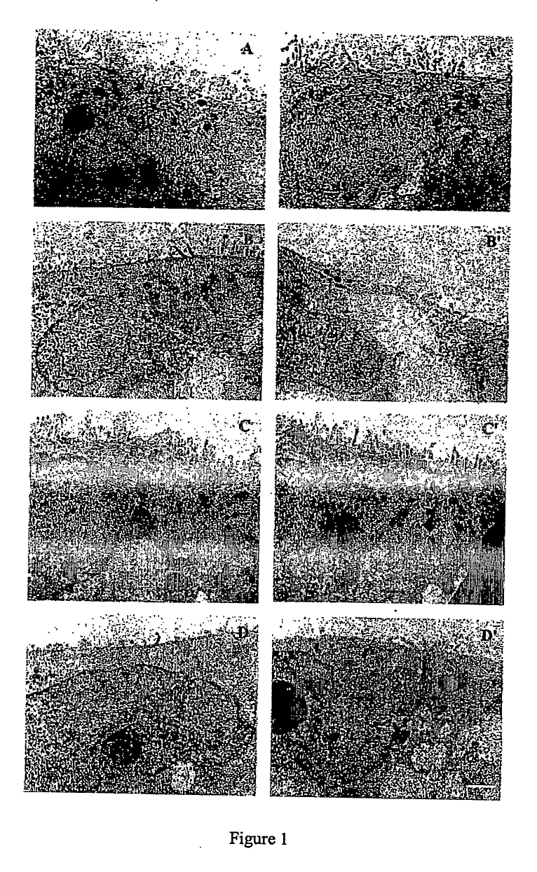

FIG. 1. Ultrastructure of confluent Caco-2 cell monolayers. Cells (7 days after seeding) were treated with drugs or vehicle for 4 days. Transmission electron micrographs show the brush-border microvilli on the apical surface of control Caco-2 cells (0.1% ethanol) (A and A′), cells treated with geraniol (400 μM) (13 and B′), cells treated with 5-FU (5 μM) (C and C′), or cells treated with geraniol and 5-FU (D and D′). Bar represents 2 μM.

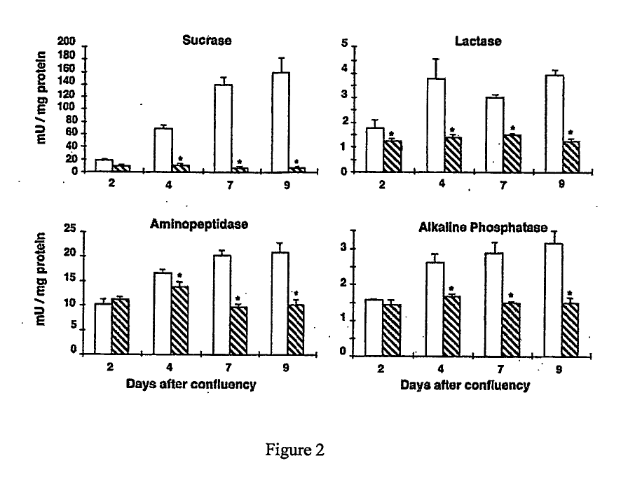

FIG. 2. Specific activity (milliunits/milligram of protein) of sucrase, lactase, alkaline phosphatase, and L-aminopeptidase in brush-border membranes isolated from Caco-2 cells. Confluent cells (7 days after seeding) were maintained for 2, 4, 7, and 9 days in culture in the absence (□) or presence ( [symbol] ) of 400 μM geraniol. The culture medium was replaced every 24 h. Data are means±S.E.M. of three separate experiments. *, for each column, p<0.05 (Student's t test).

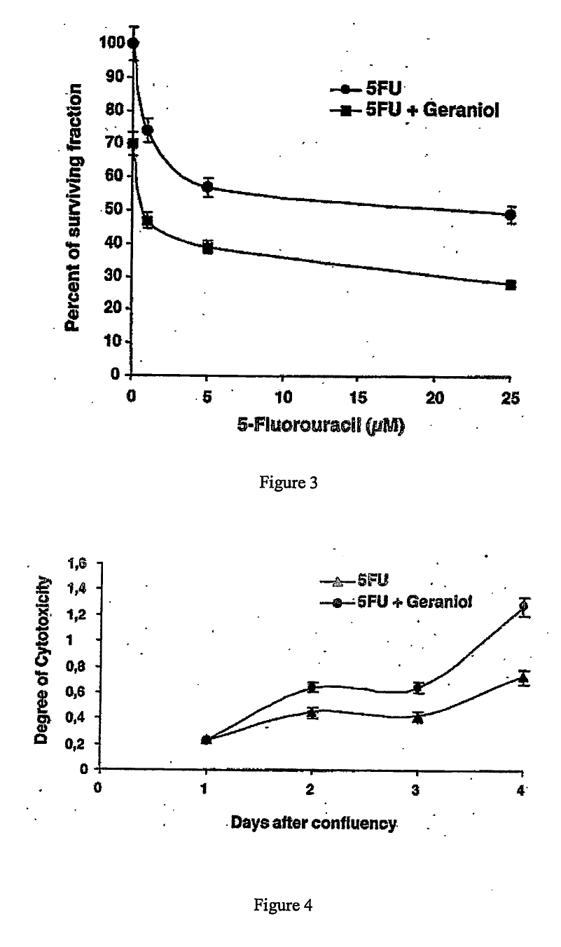

FIG. 3. Effect of geraniol in combination with increasing doses of 5-FU on Caco-2 cell growth. Confluent cells (7 days after seeding) were exposed for 8 days to 5-FU alone (●) or to 5-FU with geraniol (▪). Geraniol (400 μM) and 5-FU (5 μM) were replaced every 24 h. Values represent means S.E.M. (n=8), p<0.05.

FIG. 4. Effects of geraniol on 5-FU cytotoxicity. Cells were exposed to 5-FU (5 μM) (▴) or to geraniol (400 μM) together with 5-FU (−) between days 7 and 10 after seeding. The medium was changed every day. Cytotoxicity was assessed by determining the release of LDH into the culture medium. Data are means±S.E. (n=4), p<0.05.

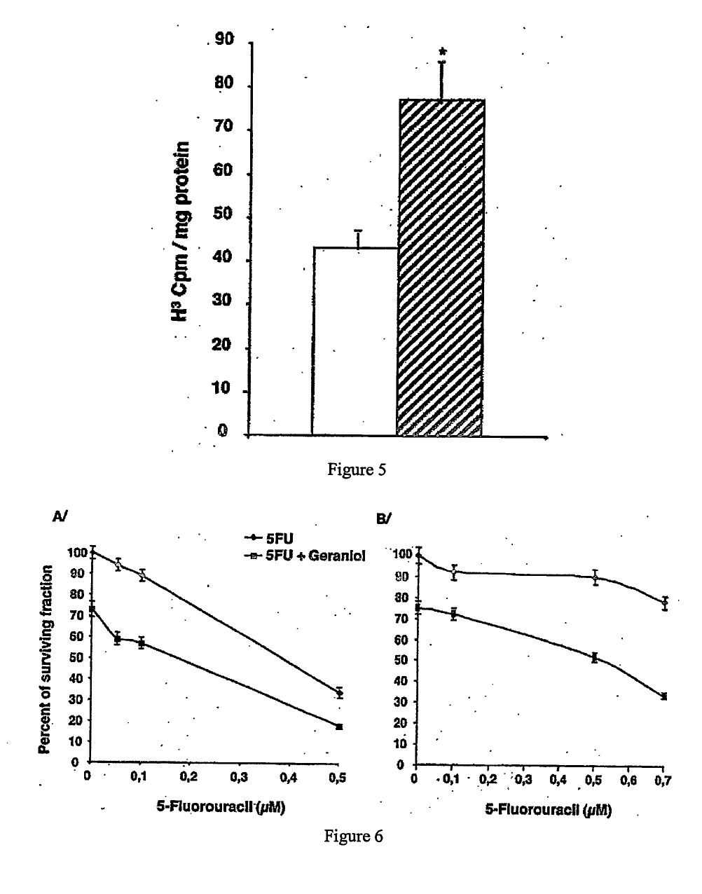

FIG. 5. Intracellular accumulation of 5-FU in the presence of geraniol. At day 7, cells were incubated for 9 h with medium containing either

5-FU (5 μM) and 1.5 μCi/ml 5-[6-³H]FU (□), or

5-FU (5 μM) and 1.5 μCi/ml 5-[6³H]FU and geraniol (400 μM) ( [symbol] ).

FIG. 6. Inhibition of Caco-2 (A) and SW620 (B) cell proliferation by 5-FU alone or together with geraniol. Cells were exposed for 8 days to 5-FU alone (♦) and various combinations of 5-FU and geraniol (▪). Geraniol and 5-FU were replaced every 24 h. Values represent means±S.E.M. (n=8), p<0.05.

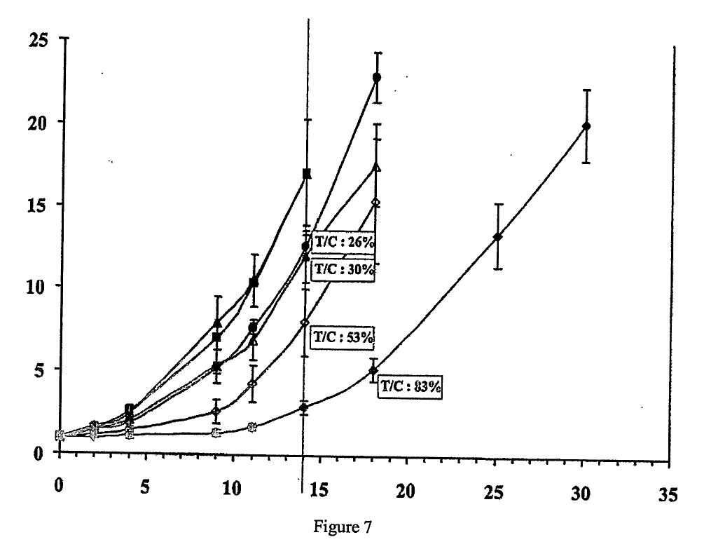

FIG. 7. Effect of combined administration of Geraniol and 5-FU on the growth of human colonic tumors TC-118: The relative tumoral volume (VTR) is indicated as a function of days after the beginning of the treatment, for control mice (▪) mice treated with 5-FU alone at 20 mg/kg/day (▴) or 40 mg/kg/day (Δ) or geraniol alone at 150 mg/kg/day (●), or geraniol at 150 mg/kg/day+5-FU at 20 mg/kg/day (⋄) or 40 mg/kg/day (♦). T/C=percentage of inhibition of the tumoral growth.

### EXAMPLE 1

### Effect of Geraniol on CACO-2 Cell Morphological Differentiation

Cell Culture.

Caco-2 and SW620 cells were obtained from the European Collection of Animal Cell Culture (CERDIC, Sophia Antipolis, France) and were cultured in 75-cm² Falcon flasks containing Dulbecco's modified Eagle's medium (DMEM) and 25 mM glucose supplemented with 10% heat-inactivated horse serum, 100 U/ml penicillin, and 100 μg/ml streptomycin. Cells were incubated at 37° C. in a humidified atmosphere of 5% CO₂ and subcultured after trypsinization (0.5% trypsin/2.6 mM EDTA). They were used for up to 30 to 40 passages.

In all experiments, cells were seeded at 6×10⁵ cells on culture dishes (100 mm in diameter), or at 4500 cells/well in 96-well plates. Caco-2 and SW620 cells were grown in DMEM supplemented with 3% horse serum, 5 μg/ml transferrin, 5 ng/ml selenium, and 10 μg/ml insulin (TSI defined medium; Invitrogen, SARL, Cergy-Pontoise, France). Geraniol (Sigma-Aldrich, Saint Louis, Mo.) was dissolved in absolute ethanol, and 5-FU (Teva Pharmachemie B.V., Mijdrecht, The Netherlands) was diluted in PBS at a final concentration of 50 mg/ml. The compounds were added to the culture medium 24 h or 7 days after cell seeding (final concentration of ethanol, 0.1%).

In all experimental conditions, culture medium, geraniol, and 5-FU were replaced every 24 h. Cells were harvested after various times, washed three times with phosphate-buffered saline (PBS) (pH 7.2), and kept at −70° C. until assays were performed.

Electron Microscopy.

Caco-2 cells were seeded on plastic coverslips in Petri dishes, and culture medium was changed every 24 h. At day 7, confluent Caco-2 cells were fixed for 2 h in sodium cacodylate buffered 2% glutaraldehyde (0.125 M, pH 7.4) at room temperature. Cells were rinsed in sodium cacodylate buffer and postfixed in 1% osmium tetroxide in the same buffer for 2 h⁻ at room temperature, and then washed overnight. They were subsequently dehydrated in graded ethanols and embedded in Spurr resin by classical methods (Spurr, 1969). Ultrathin sections post-stained with 2% uranyl-acetate were observed at 60 kV with a Hitachi H-7500 transmission electron microscope, and pictures were obtained using the Advantage CCD camera system of AMT (Advanced Microscopy Techniques Corp., Danvers, Mass.).

Results

Caco-2 cells undergo phenotypic changes after confluency, which are characterized by an enterocytic morphology and by the expression of various hydrolases in the brush-border membrane typical of the differentiated state.

At day 4 after confluency (7 days after seeding), cells form a monolayer showing apical microvilli and tight junctions characteristic of the differentiated state. Brush-border microvilli were numerous, and they were long in nontreated Caco-2 cells (FIG. 1, A and A′). After treatment with geraniol (400 μM), the brush border was modified; microvilli were scarce, and they were shorter (FIG. 1, B and B′). Treatment with 5-FU (5 μM) alone did not modify brush-border membranes (FIG. 1, C and C′). Microvilli at the apical surface were short, swollen, and scarce in cells treated with 5-FU (5 μM) together with geraniol (400 μM), and cells had irregular nuclei with condensed chromatin (FIG. 1, D and D′).

Note that geraniol appears to reduce the length and density of microvilli. Nuclei have a very invaginated aspect after the combined treatment.

### EXAMPLE 2

### Effect of Geraniol on CACO-2 Cells Functional Differentiation

Isolation of Brush-Border Membranes and Hydrolase Assays.

Caco-2 cells were homogenized in 4 ml of Tris-mannitol buffer (50 mM mannitol, 2 mM Tris, pH 7.1) by sonication. Brush-border membranes were isolated as described by Schmitz et al. (1973).

Sucrase activity was determined according to Messer and Dahlgvist (1966) and lactase activity according to Koldovsky et al. (1969). Alkaline phosphatase activity was assayed by the method of Garen and Levinthal (1960), and N-aminopeptidase activity was determined according to Maroux et al. (1973).

Enzyme activities were expressed as specific activities (milliunits/milligram of protein), 1 U of activity corresponding to 1 μmol Of product formed/min at 37° C.

Results

The treatment of Caco-2 cells at confluency (7 days after seeding) with 400 μM geraniol inhibited the increase of sucrase and lactase activities normally observed at this stage (FIG. 2). The inhibition was approximately 90% for sucrase and 70% for lactase. In addition, geraniol also significantly inhibited the increase in alkaline phosphatase and aminopeptidase activities by about 50%. In fact, the level of all the differentiation markers (brush-border hydrolases) remained at their initial level measured at day 7 after plating, just before the treatment with geraniol.

### EXAMPLE 3

### Effect of Geraniol and 5-FU on CACO-2 Cell Growth

Cell Growth.

Cells were seeded in 96-well plates and incubated for different times. Cell growth was stopped by addition of 50 μl of trichloroacetic acid (50%, v/v), and the protein content of each well was determined by staining with sulforhodamine B (Skehan et al., 1990): Absorbance was determined at 490 nm. The relationship between cell number (protein content per well) and absorbance was found to be linear from 0 to 200,000 cells/well.

The effects of increasing doses of 5-FU alone or in combination with geraniol were determined after treatment for 8 days. The concentration of 5-FU ranged between 1 and 25 μM; geraniol was used at its IC₃₀ value (400 μM). Geraniol and 5-FU were replaced every 24 h.

Statistical differences between control and geraniol-treated cells were evaluated using the Student's t test. Differences were considered to be significant for values of p<0.05.

Results

As shown in FIG. 3, the anti-proliferative effects of 5-FU were significantly enhanced in the presence of geraniol. Geraniol alone provoked a 30% cell loss, and treatment with 1 μM 5-FU alone caused a cell loss of 25%. When combined, 5-FU (1 μM) and geraniol caused a 55% cell loss. Similarly, at higher concentrations of 5-FU, the number of surviving cells was reduced by half in the presence of geraniol.

The results of Table I below show that when geraniol was added to the culture medium, the amount of 5-FU required to reach the IC₅₀ value was significantly reduced. Thus, with 5-FU alone, the amount of 5-FU necessary was 25 μM and this amount was reduced to 1 μM in the presence of geraniol.

TABLE 1

| | Cell Line | | |
|---|---|---|---|
| | Caco-2 | | |
| IC₅₀ 5-FU (μM) | Confluent | Growing | SW620 |
| 5-FU alone | 25 ± 0.04 | 0.4 ± 0.009 | 2 ± 0.1 |
| 5-FU + geraniol (IC₃₀) | 1 ± 0.05 | 0.2 ± 0.007 | 0.5 ± 0.22 |

Values represent means ± S.E.M. (n = 8), p < 0.05.

### EXAMPLE 4

### Effect of Geraniol on CACO-2 Cell Death

To determine whether geraniol caused apoptosis, DNA fragmentation assays and annexin V labeling were performed.

Determination of Apoptosis and Cytotoxicity.

Caco-2 cells (7×10⁵ cells/10-ml Petri dish) were seeded and treated with 5-FU (5 μM) alone or together with geraniol (IC₃₀: 400 μM) and 5-FU for 24 h, at day 7. After trypsinization, cells were collected by centrifugation and stored at −80° C. Apoptotic DNA was separated from genomic DNA, using the Suicide Track DNA ladder kit (Oncogene Research Products, Cambridge, Mass.). DNA fragments were separated by electrophoresis and stained with ethidium bromide.

Apoptosis was further tested by evaluating phosphatidylserine membrane externalization by measuring annexin V-conjugated fluorescein isothiocyanate (FITC) binding using an Annexin V-FITC kit (Medsystems Diagnostics GmbH, Vienna, Austria). Briefly, cells were washed in cold PBS without calcium and, for each sample, 5×10⁵ cells were resuspended in 100 μl of reaction buffer (10 μl of binding buffer 10-fold concentrated, 10 μl of propidium iodide, 1 μl of annexin V-FITC, and 79 μl of deionized water. After incubation for 15 min in the dark at room temperature, each sample was diluted with binding buffer to obtain an appropriate final volume for flow cytometry.

To determine cytotoxicity, cells (4×10⁵/well) were seeded on culture dishes (100 nm diameter) and incubated in DMEM culture medium supplemented with 3% FCS. On the 7^{th} day after seeding, cells were incubated with 5-FU (5 μM) alone or together with geraniol (IC₃₀: 400 μM) and 5-FU for 1, 2, 3, and 4 days. Then, cytotoxicity was assessed by determining the release of lactate dehydrogenase (LDH) (Skehan et al., 1990) into the culture medium using the Cyto Tox R nonradioactive cytotoxicity assay kit (Promega, Madison, Wis.).

Results

Exponentially growing cells and confluent cells (7 days after seeding) treated with 5-FU (5 μM) with or without geraniol for 24 h did not exhibit apoptotic DNA fragmentation ladders (results not shown). These results were confirmed by the absence of annexin V labeling using flow cytometry analysis (results not shown). These results show that 5-FU treatment induced nonapoptotic cell death characterized by the condensation of nuclear chromatin, cytoplasmic vacuolation, and absence of annexin staining or DNA fragmentation (Sperandio et al., 2000).

The effect of geraniol on 5-FU cytotoxicity was assessed after confluency (7 days after seeding) by measuring LDH release in the culture medium after 4 days of treatment with the drugs. As shown in FIG. 4, the presence of geraniol, which alone showed no cytotoxic effect, enhanced the cytotoxic effects of 5-FU (5 μM) by a factor of 2.

### EXAMPLE 5

### Effect of Geraniol on Cellular Uptake of 5-FU

Measure of 5-FU Uptake.

Intracellular accumulation of 5-FU (5 μM) in cells treated with or without geraniol (400 μM) was determined by measuring the amount of 5-[6-³H]FU (1 mCi/μmol; specific activity: 318.2 GBq/nmol; PerkinElmer Life Sciences, Boston, Mass.), taken up by Caco-2 cells. Approximately 5×10⁵ cells were seeded in culture dishes (100 mm diameter) and incubated at 37° C. for 24 h. The culture medium was replaced every 24 h. At day 7 after seeding, when cells reached confluency, they were incubated for 9 h in a culture medium containing 5-FU (5 μM) and 1.5 μCi/ml [6-³H]FU with or without geraniol. Cells were then collected, washed three times with cold PBS, and sonicated. The radioactivity present in the trichloroacetic acid-precipitable fraction was determined by liquid scintillation spectrometry.

Results

Intracellular accumulation of 5-[6-³H]FU, determined in the presence or absence of geraniol after 9 h of treatment, showed (FIG. 5) that the uptake of 5-FU by Caco-2 cells was enhanced 2-fold in the presence of geraniol.

### EXAMPLE 6

### Effect of Geraniol on Growing CACO-2 and SW620 Cells Treated with 5-FU

The comparative effects of geraniol and 5-FU were studied on exponentially growing Caco-2 and SW620 cells. Cells were cultured as described in Example 1.

Evaluation of Cell Resistance to 5-FU Treatment.

To evaluate cell resistance to 5-FU treatment, we tested increasing doses of 5-FU on both differentiated and exponentially growing Caco-2 cells, and also on growing SW620 cells (a nondifferentiating colon cell line). Table 1 shows that confluent cells were more resistant than exponentially growing cells. For confluent Caco-2 cells, the IC₅₀ for 5-FU was 25 μM, whereas for growing Caco-2 and SW620 cells the IC₅₀ values for 5-FU were, respectively 0.4 and 2 μM.

Results

For growing Caco-2 cells, the concentration of geraniol (200 μM) necessary to reach the IC₃₀ value was reduced 2-fold compared with cells at confluency (400 μM). The effects of graded doses of 5-FU alone or in combination with geraniol were determined. Cells were treated for 8 days with geraniol at its IC₃₀ value (200 μM) 24 h after seeding. For both cell lines, geraniol potentiated the inhibition of cell growth observed with 5-FU alone (FIG. 6). As illustrated in Table 1, growing SW620 cells were more resistant to 5-FU treatment than proliferating Caco-2 cells (IC₅₀ for 5-FU: 2 and 0.4 μM, respectively). In the presence of geraniol, the sensitivity of growing cells to 5-FU treatment was also significantly increased. Furthermore, measurement of CI (mean±S.E.) at the IC₅₀ iso-effect level, which determines whether the interactions of the two drugs are synergistic, additive, or antagonistic, indicated that the association of 5-FU and geraniol presented a synergistic effect on Caco-2 cells (CI=0.9±0.014), whereas the effect appeared to be additive for SW620 (CI=0.97±0.10).

### CONCLUSION

The above results show that geraniol sensitizes human colonic cancer cells to 5-FU treatment. The two cell lines, Caco-2 and SW620, used in the present study, differ in their sensitivity to 5-FU treatment, and they also responded differently to geraniol and to the combination of 5-FU with geraniol. The effect of geraniol is greater on differentiated Caco-2 cells which are also the more resistant to 5-FU than on undifferentiated Caco-2 and SW620 cells.

It also appears that geraniol acts on two major targets involved in the resistance of colon cancer cells to chemotherapeutic agents: the process of cell differentiation and membrane permeability to the drug. Interaction of geraniol with the cell membrane prevents the differentiation process and further appears to facilitates the uptake of the chemotherapeutic agent by cancer cells.

This can permit the use of lower concentrations of chemotherapeutic drugs and, at the same time, lower their secondary effects.

### EXAMPLE 7

### Effect of Combined Administration of Geraniol and 5-FU on the Growth of 5-FU-Resistant Colonic Tumour

The effect of geraniol and 5-FU were evaluated in vivo on the growth of 5-FU-resistant human colonic tumors (TC-18) transplanted in Swiss nu/nu mice.

TC 118 is the hepatic metastasis of a primary human colorectal tumor is used as xénogreffe. Fresh fragments of the tumor are implanted in the subcutaneous tissue of female Swiss nulnu mice of from 5 to 6 weeks.

The appearance of the tumor is watched by a bi-weekly exam of mice and the time of appearance is registered. Tumors are detected by palpation as soon as they measure approximately 3×3 mm (that is approximately 15 mm³). The growth of tumors is measurable in a reliable way from a diameter of 5×5 mm (60 mm³). Tumors are measured with a calliper rule, 3 times a week, until the sacrifice of the animal. Two perpendicular diameters are measured and the volumes of tumors are calculated according to the following equation:
V=A×B²/2

where A represents the large diameter and B the small diameter.

The relative tumoral volume (VTR) is calculated according to the formula:
VTR=Volume at day D/Initial volume

The active principles were administered to mice when tumors reached a diameter of 5-8 mm (60 to 250 mm³). Geraniol and 5-FU were administered every day during 5 days by intraperitoneal way in a volume of 0,2 ml of NaCl 0.9%. The doses were of 150 mg/kg/j for geraniol and 20 mg/kg/day or 40 mg/kg/day for 5-FU. When these molecules were given in combination, they were injected simultaneously. A control group was treated with excipient (NaCl 0.9%).

The mean value of the VTR was calculated for every group on every day corresponding to the measure of tumors.

The curves of tumoral growth were obtained in every experimental group or for every individual tumor by reporting the values of the VTR as a function of time (expressed in days after the beginning of the treatment). The measures were individually recorded, allowing a follow-up of the tumoral growth.

Mice were sacrificed as soon as the tumor reached a volume of 2500 mm³. Mice not presenting tumors were sacrificed at the same time.

The percentage of inhibition of the tumoral growth was calculated at day 14 for the various treatments, according to the formula:
(VTR of treated mice/VTR of control mice)×100.
Results

The results are shown on FIG. 7.

The combined intraperitoneal administration of 5-FU (20 mg/kg/day) and geraniol (150 mg/kg/day) caused a 53% inhibition of the tumoral growth after 14 days compared to an inhibition of 26% with geraniol alone. 5-FU alone at 20 mg/kg/day had no effect on the development of the tumour. When 5-FU alone is administered at 40 mg/kg/day, an inhibition of 30% of the tumoral growth is observed, while a combination of 5-FU at 40 mg/kg/day and geraniol at 150 mg/kg/day induces a 83% inhibition.

These results show that that the combined administration of geraniol and 5-FU sensitizes in vivo human colonic tumours to 5-FU treatment.

### EXAMPLE 8

### Effect of Geraniol and CPT-110N CACO-2 Cell Growth

The effect of various doses of CPT-11 alone or in combination with geraniol was determined after 8 days of treatment of Caco-2 cells. Concentrations of CPT-11 of from 0.2 to 0.8 μM were tested and geraniol was used at a concentration which induces a 10% inhibition of the cell growth (IC₁₀).

As shown in Table II, the effect of CPT-11 is significantly increased in the presence of geraniol. The concentration of CPT-11 which induces an inhibition of 50% of the growth (IC₅₀) is of 0.7 μM while the IC₅₀ is of 0.4 μM for the association of geraniol

TABLE II

| | | IC₅₀ (μM) |
|---|---|---|
| IC₅₀ CPT-11 | CPT-11 seul | 0.7 ± 0.085 |
| | CPT-11 + Geraniol (IC₁₀) | 0.4 ± 0.054 |
| IC₅₀ Geraniol | Geraniol seul | 200 ± 0.03 |
| | Geraniol + CPT-11 (IC₁₀) | 87 ± 0.017 |

### REFERENCES

Burke Y D, Stark M J, Roach S L, Sen S E and Crowell P L (1997). Lipids 32: 151-156 Carnesecchi S, Langley K, Exinger F, Gosse F, and Raul F (2002). J Pharmacol Exp Ther 301:1-6.
Carnesecchi S, Schneider Y, Ceraline J, Duranton B, Gosse F, Seiler N, and Raul F (2001). J Pharmacol Exp Ther 298:197200.
Carnesecchi S, Schneider Y, Ceraline J, Duranton B, Gosse F, Seiler N, and Raul F (2001). J Pharmacol Exp Ther 298:197200.
Elson C E and Yu S G (1994). J Nutr 124:607-614.
Garen A and Levinthal C (1960). Biochim Biophys Acta 38:470-483.
Glaise D, Ilyin G P, Loyer P, Cariou S, Bilodeau M, Lucas J, Puisieux A, Ozturk M, and Guguen-Guillouzo G (1998). Cell Growth Differ 9:165-176.
Ho S B, Pei-Sha Y, Rajvir D, Brent A, Neushwander-Tetri Y, Basbaum C, and Young S (1994). J Cell Physiol 160:213-226:
Koldovsky O, Asp N G, and Dahlqvist A (1969). Anal Biochem 27:409-418.
Lesuffleur T, Violette S, Vasile-Pandrea I, Dussaulx E, Barbet A, Muleris M, and Zweibaum A (1998). Int J Cancer 76:383-392.
Lesuffleur T, Kornowski A, Augeron C, Dussaulx E, Barbat A, Laboisse C, and Zweibaum A (1991). Int J Cancer 49:731-737.
Lotan R (1990). Cancer Res 50:3453-3464.
Mariadason J M, Velcich A, Wilson A J, Augenlicht L H, and Gibson P R (2001). Gastroenterology 120:889-899.
Maroux S, Louvard D, and Baratti J (1973). Biochim Biophys Acta 321:282-295.
Messer M and Dahlqvist A (1966). Anal Biochem 14:376-392.
Pandrea I V, Carrière V, Barbat A, Cambier D, Dussaulx E, Lesuffleur T, Rousset M, and Zweibaum A (2000). Exp Mol Pathol 69:37-45.
Pinto M, Robine-Leon S, Appay M D, Kedinger M, Haffen K, Fogh J, and Zweibaum A (1983). Biol Cell 47:323-330.
Rousset M (1986). Biochimie 68:1035-1040.
Schmitz J, Preiser H, Maestracei D, Ghosh B K, Cerda J J, and Crane R K (1973). Biochim Biophys Acta 323:98-112.
Shoff S M, Grummer M, Yatvin M B, and Elson C E (1991). Cancer Res 51:37-42.
Skehan P, Storen R, Scudiero D, Monks A, McMahon J, Vistica D, Warren J T, Boekesch H, Kenney S, and Boyd M R (1990). J Natl Cancer Inst 82:1107-1112.
Sperandio S, De Belle I, and Bredesen D E (2000). Proc Natl Acad Sci USA 97:14376-14381.
Spurr A R (1969). J Ultrastruct Res 26:39.
Yu S G, Hildebrandt L A, and Elson C E (1995). J Nutr 125:2763-2767.

1) The use of geraniol for preparing a therapeutic composition for blocking the development and differentiation of tumor cells.

2) The use of claim 1, wherein geraniol is used for preparing a therapeutic composition potentiating the cytotoxic effect of an antitumoral agent.

3) The use of any of claims 1 or 2, wherein said tumor cells are selected among colorectal tumor cells, hepatoma cells, digestive or aerodigestive cancer cells, prostate cancer cells.

4) The use of claim 2, wherein said antitumoral agent is selected from 5-fluorouracil, oral derivatives of fluorouracil, irinotecan (CPT-11), platinum diammonocyclohexan (oxaliplatinium), and taxol.

5) A composition comprising a cytotoxic antitumoral agent and geraniol.

6) The composition of claim 5, wherein said antitumoral agent is selected from 5-fluorouracil, oral derivatives of fluorouracil, irinotecan (CPT-11), platinum diammonocyclohexan (oxaliplatinium), and taxol.
